(19) United States
(12) Patent Application Publication
(10) Pub. No.: US 2002/0098553 A1
(43) Pub. Date: Jul. 25, 2002

(54) Process for producing carrageenan with reduced amount of insoluble material

(76) Inventors: Pierre-Etienne Bost, Paris (FR); Andres Hohlberg Recabarren, Santiago (CL); Jaime Zamorano Palma, Puerto Montt (CL)

Correspondence Address: Jean-Louis Seugnet, Rhodia Inc., Bldg. N-2, 259 Prospect Plains Road, Cranbury, NJ, 08512-7500, US

(21) Appl. No.: 09/766,038

(22) Filed: Jan. 19, 2001

(51) Int. Cl. C12P 19/04; C07H 13/12

(52) U.S. Cl. 435/101; 536/118

(57) ABSTRACT

The invention relates to a process for producing carrageenans, more particularly kappa and iota carrageenans, containing less than 2% by weight of insoluble material, comprising the steps of preparing an aqueous suspension of a seaweed which contain carrageenans and treating the resultant suspension with one or a mixture of enzyme(s).

The present invention relates to a process for producing carrageenans, more specifically kappa and iota carrageenans, containing reduced amount of insoluble material.

In particular, the invention relates to a process for producing carrageenans, more particularly kappa and iota carrageenans, containing less than 2% by weight of insoluble material, comprising the steps of:

i—preparing an aqueous suspension of a seaweed which contain carrageenans;

ii—reacting the resultant suspension with one or a mixture of enzyme(s).

Carrageenans are complex mixtures of sulphated polysaccharides comprising linear polymers of 1,3 bound β-D-galactose units and of 1,4 bound α-D-galactose units. Different types of carrageenans such as kappa, iota, lambda are differentiated by the sequence of their galactose units and by the degree of substitution in such units.

Different types of carrageenan are obtained from different species of seaweed. Kappa carrageenan, for example, is produced predominantly by red seaweeds such as Eucheuma cottonii, Chondrus crispus, Gigartina stellata, Gigartina skottsbergii, Gigartina radula, Furcellaria fastigata and Hypnea spp. While Iota carrageenan, for example is produced by Eucheuma spinosum and Gymnogongrus furcellatus.

It constitutes the principal structural component of the seaweed and it is located in the cell walls as well as in the intercellular matrix of the plant.

Kappa and iota carrageenans have valuable properties as a food additives and are widely used as emulsifying, gelling, thickening, and suspending agents. For similar type of purposes, kappa and iota carrageenans have also been frequently used in home and personal care products.

Kappa carrageenan tends to form strong rigid gels. However, kappa carrageenans obtained from different sources vary somewhat: for example the kappa from Eucheuma cottonii produces a higher gel strength and somewhat more brittle gels than the kappa from Chondrus crispus or Gigartina sp.

When extracting carrageenans from seaweeds, specifically kappa and iota carrageenans from red seaweeds, residual insoluble solids of organic matter such as cellulose, hemicellulose, beta-glucans, proteinaceous and lipoidal components and other polymeric materials present in the cell wall and/or in the intercellular matrix will remain in the medium. The insoluble material usually represents 6 to 15% by weight of the dry matter of the seaweed. If not removed during the production of the carrageenan, these contaminating materials will influence the color, appearance, taste and smell of the final product in which carrageenans, more specifically kappa and iota carrageenans, are later employed.

By insoluble material we means Acid Insoluble Matter like in the JECFA specification for INS 407, wich is mainly cellulosic material See G. O. Phillips, 1996. “The chemical identification of PNG-carrageenan” In: Gums and Stabilisers for the Food Industry 8. G. O. Phillips, P. A. Williams and D. J. Wedlock (Eds) IRL Press, pp.403-421.

Depending on the final application of the carrageenans, the residual amount of insoluble materials may be a more or less important issue.

The traditional process for the production of <<purified carrageenan>> comprises extraction of carrageenan from fresh or dried seaweed in hot water at a basic pH. The aqueous extract, which contains about 1% carrageenan, is clarified usually through filtration to remove insoluble material (cellulose, hemicellulose, residual organic material, etc.). The filtered extract, which optionally can be concentrated to about 4% and subjected to various purification treatments such as filtering with activated carbon, bleaching, etc. is then treated with an alcohol or with a salt to precipitate the carrageenan.

Purified carrageenan is typically colorless, tasteless, odourless, and will create a non-opaque gel in water. Such carrageenans are generally of a quality suitable for pharmaceutical applications, and any other application where product clarity and lack of odor and taste are primary considerations.

The production of purified carrageenans requires high energy consumptions and may involve substantial environmental pollution and therefore, several attempts have been made to provide less costly carrageenans known as <<semi-refined carrageenan>>.

Semi-refined carrageenans are usually prepared by heat-treating whole seaweed without involving filtration or any other form of clarification in alkaline solutions under conditions, which modify the carrageenan by at least partially removing sulphate groups. Carrageenans of this type are generally more economical to produce. However The absence of the filtration or the clarification step will lead to the obtention of semi-refined carrageenans containing residual organic material which influences the color, taste and smell of the product in which it is used.

Due to presence of high levels of insoluble plant materials, final products containing this type of carrageenan will have a cloudy appearance and will create a gel appearance that may not be desirable in many applications. Consequently, the use of semi-refined carrageenans is limited to a smaller range of applications in instances where impurities can be tolerated, where clarity and smoothness of solution gels are not required, and where production cost considerations are of singular importance.

An object of the invention is to produce carrageenans, particularly kappa and iota carrageenans, having a high degree of purity using a low cost process, which implies not clarification like previously described.

Another object of the present invention is to provide a process that removes efficiently the contaminating materials present in seaweed which contain carrageenans, and preserves at the same time the carrageenan and its properties.

Accordingly, the present invention provides, in one aspect, a process for producing carrageenans, more particularly kappa and iota carrageenans, containing less than 2% by weight of insoluble material, comprising the steps of:

i—preparing an aqueous suspension of a seaweed which contain carrageenans;

ii—reacting the resultant suspension with one or a mixture of enzyme(s).

The process according to the invention gives a higher yield of <<purified carrageenan>> relative to the known processes for preparing such, since substantial loss of insoluble materials occurs without substantial loss of carrageenan (preferably kappa and iota).

The significant reduction of insoluble materials in the carrageenans obtained as a result of the process according to the invention, makes them comparable to known commercially available <<refined carrageenans>>.

A further advantage is that carrageenans obtained according to the process of the invention are suitable for the preparation of water gels with improved appearances.

Another advantage of the present invention is its low cost compared to the known processes for preparing <<purified carrageenans>>.

Other objects, advantages, features and characteristics of the present invention will become more apparent upon consideration of the following detailed description, examples and the claims.

As mentioned above, the process of the invention for producing carrageenans, more particularly kappa and iota carrageenans, containing less than 2% by weight of insoluble material, comprising the steps of:

i—preparing an aqueous suspension of a seaweed which contain carrageenans;

ii—reacting the resultant suspension with one or a mixture of enzyme(s).

The improvement of the degree of purity (less than 2% by weight of insoluble material) of carrageenans in this process is mainly due to the use of enzyme(s).

The seaweed employed as the starting material may be chosen among Eucheuma cottonii, Eucheuma spinosum, Chondrus crispus, Gigartina stellata, Gigartina skottsbergii, Gigartina radula, Gymnogongrus furcellatus, Furcellaria fastigiata and Hypnea spp. Preferably, the seaweed is Eucheuma cottonii, Gigartina radula, Gigartina skottsbergii.

In one embodiment, dried seaweed containing carrageenan is blended with a liquid, preferably aqueous, to form the aqueous suspension of step (i). A mechanical agitation can be used.

In another embodiment, the aqueous suspension may be obtained after:

washing and sorting of a raw seaweed which contain carrageenans; and

optionally, chopping and/or bleaching the said seaweed.

In the production of carrageenans, these operations are typically performed and are thus known to a person skilled in the art.

In general, washing enables sand and other particulates to be loosened and released from the raw seaweed.

The seaweed which contain carrageenans may be washed, for instance, with an aqueous saline wash solution of sodium or potassium chloride, preferably at a temperature of about 25 to 30° C.

The washed seaweed is subsequently sorted. Sorting generally refers to the removal of plant materials other than the seaweed that is desired for processing, such as the removal of other undesired seaweeds, ties used to fix the seedling seaweed to an underwater cultivation system, other bits of debris collected from the beach and water during harvest.

Sorting may also result in the separation of the different phases existing in the history lifes of the source, which, for certain species, contain different types of carrageenans.

Sorting may be performed using chemical or physical methods, such as resorcinol identification of Kappa and iota carrageenans and/or optical detection of shape differences between the different types of seaweed and pneumatic separation over a belt conveyor. Sorting may be also performed manually.

Optionally, the washed and sorted seaweed may be chopped into shorter lengths prior to further processing. Chopping increases the surface area available for reaction and improves homogeneity of the reaction mixture and ultimately accelerates the reaction progress.

According to the improvements of the present invention, it is preferable to chop the seaweed into pieces of approximately 5 to 50 cm², and preferably of 10 to 30 cm² in order to reduce process times by exposing an increased seaweed surface area to the subsequent enzyme treatment.

A hammermill or grinding knives may be used for this purpose.

The washed and sorted seaweed, either directly or after chopping, may be optionally subjected to bleaching.

Bleaching results in the oxidation of pigments (such as chlorophyll, phycoeritrin, phycocianin, beta-carotene and ceaxhantin) that impart undesired color to the end product.

Bleaching may be performed by any suitable oxidizing agent, such as hydrogen or sodium peroxide, sodium or calcium hypochlorite, sodium dichloroisocyanurate, boric acid, ozone, chlorine dioxide, oxygen.

In either embodiment, the content of seaweed dry matter in the suspension of step (i) is in the range of 5 to 20% by weight and preferably in the range of 10 to 15% by weight expressed by weight to weight.

The formed suspension facilitates later mixing with the enzyme(s).

As already mentioned, the improvement of the degree of purity of carrageenans and more specifically kappa and iota carrageenans (less than 2% by weight of insoluble material) in the process of the invention is essentially due to the specific action of enzyme(s) used.

Enzymes are widely known and applied in industrial processes. Due to their efficiency, specific action, the mild conditions in which they work and their high biodegradability, enzymes are very well suited to a wide range of industrial applications. Moreover, industrial processes using enzymes are potentially energy saving and save investing in special equipment resistant to heat, pressure or corrosion.

However, finding a suitable enzyme or mixture of enzymes for a desired transformation or with a defined specificity is generally difficult.

Moreover, since an enzyme will interact with only one type of substrate or group of substrates to catalyze a certain kind of transformation, in certain circumstances more than one enzyme may be necessary.

The present invention discloses the use of enzymes capable of attacking cellulose, hemicelluloses and other polymeric materials in the seaweed. More specifically, the present invention discloses the use of enzymes having cellulase and/or hemicellulase activities in the removal of the contaminant material present in the seaweed. “Cellulase” refers to a complete cellulase system that contains any and all cellobiohydrolase proteins, endoglucanase proteins and β-glucosidase proteins. “Hemicellulase” refers to enzymes involved in the hydrolysis of hemicelluloses—non-cellulosic cell wall polysaccharides. “Xylanase” refers to a complete hemicellulase system that is involved in the breakdown of heteroxylans and contains, but is not limited to, any and all endo-1,4-β-xylanase proteins, β-xylosidase proteins, α-L-arabinofuranosidase proteins and esterase proteins.

As previously mentioned, in the process of the present invention enzymes can be added alone or in combination with others. Preferably, mixtures of enzymes comprising cellulase and/or xylanase activities are used in the step (ii).

For instance, such enzyme mixtures may be obtained from fungal strains of Trichoderma, Aspergillus, or Penicillium. The mixtures are isolated from the growth medium of these microorganisms without further purification. It should be noted that the activities and ratios of the different enzymes in mixtures depend on the substrate, the growth conditions and the microbial strains used in fermentation. It should also be noted that the present invention is by no means limited to these microorganisms.

If the mixture so isolated contains the desired range and ratio of enzyme activities, the mixture is used as such. The desired range and ratio of enzyme activities depends on the substrate, which has to be degraded, and is preferably determined for every substrate.

If the mixture that is isolated without further treatment does not contain the desired range and ratio of enzyme activities, mixtures from different cultures are used.

It is also possible to mix culture fluids from growth of different microbial strains or species.

Alternatively, in order to obtain the desired enzyme mixture, the enzymes are purified.

Preferably, the mixtures of enzymes comprising cellulase and/or xylanase activity are obtained by mixing the purified enzymes in predetermined amounts or by combining mixtures with predetermined activity giving the desired final enzymatic activity ratios.

The enzyme(s) are added in an amount sufficient to remove efficiently the contaminant materials.

It has been observed that the different enzymes have a synergistic effect in the degradation of the contaminant materials when used as a mixture of cellulase and/or xylanase activities in specific ratios. The preferred mass ratios of the enzymes will depend on the contaminant material to be degraded used.

The enzymes, alone or in combination, having cellulase and/or xylanase activities can be added in an amount up to about 15% by weight based on the weight of the seaweed which contain carrageenans. In most applications, the enzyme(s) added in an amount of about 5% by weight based on the weight of the seaweed which contain carrageenans will be sufficient to remove the insoluble materials contained within the carrageenan. Adding the enzymes in excess, however, has been found to cause no adverse affects. Consequently, the enzymes may be added in much greater amounts than as described above if desired.

The enzymes, cellulase or xylanase are commercially available as a liquid concentrate or as a dry powder or as granules. Any form may be used in the process of the present invention. The commercially available cellulase can be Econase CEPI (Rhöm enzyme Finland oy) or Multifect Cellulase 300 (Genecor International Inc.) and commercially available xylanase can be Econase HCP4000 (Rhöm enzyme Finland oy) or Multifect Xylanase (Genecor International Inc.).

It has been found that the enzyme catalysed degradation of proceeds at a suitable rate at room temperature. If desired, the temperature can be increased or decreased in order to increase or decrease the rate of reaction. High temperatures that will cause the enzymes degradation, known to one skilled in the art, should be avoided.

For optimal activity of the enzymes, it is preferable that the reaction takes place at a pH of more or equal than 4 and no more than 6, more preferably between 4.5 and 5.5.

After the enzymatic treatment, the aqueous suspension of step (ii) is further subjected to alkali treatment.

Alkali treatment is accomplished with an aqueous solution of a base so as to cause desulfation at the position 6 of the β 1-4 linked galactose units of the carrageenan and so as to create recurring 3,6 anhydrogalactose polymers by dehydration and reorientation. Another consequence of the alkali treatment is that it denatures the enzymes residual activities.

The base used for this step may suitably be a hydroxide or carbonate of an alkali metal, an alkaline earth metal or ammonium, for instance sodium hydroxide, potassium hydroxide, barium hydroxide calcium hydroxide, magnesium hydroxide, sodium carbonate, potassium carbonate, barium carbonate, calcium carbonate, magnesium carbonate, ammonium hydroxide, or ammonium carbonate; an alkali metal alcoholate, for example sodium methoxide, sodium ethoxide or sodium isopropoxide; a basic inorganic phosphate or tripotassium phosphate; or a quaternary ammonium hydroxide, for example tetramethyl ammonium hydroxide, trimethylethyl ammonium hydroxide, tetrabutyl ammonium hydroxide or tetraethyl ammonium hydroxide. A combination of one of the above bases may also be used.

The resulting product is then rinsed, neutralized, and optionally re-bleached. It may further be subjected to the steps of washing, dewatering, drying and finally sized preferably through grinding, the latter step being optional. These operations are typically performed in the production of carrageenans, and are thus known to a person skilled in the art.

Dewatering removes a large quantity of water from the suspension of treated carrageenans. The water content after dewatering is reduced up to approximately 50 to 70% by weight to weight.

The dewatered carrageenan is subsequently dried. Procedures for drying include, but are not limited to, continuous dryer using direct hot air, fluid bed drying using, for example, hot air at a temperature of about 90°, or by conventional air drying at, for example, 40 to 60° C.

Advantageously, the carrageenan is dried to a dry matter content of at least 80% by weight, preferably at least 85% by weight, and more preferably at least 90% by weight.

After drying, further processing of the carrageenan will generally depend on the final application. For example, the product may be dry chipped and/or milled (ground) to a specific particle size. The average particle size may be less than 200 μm, preferably less than 150 μm, and more preferably less than 75 μm.

In some instances, granules of carrageenans may also be prepared.

A second aspect of the invention relates to carrageenans, more particularly kappa and iota carrageenans, containing less than 2% by weight of insoluble material obtained by a process according to the instant invention. Such carrageenans comply with current standards for use in food stuffs.

A third aspect of the present invention relates to the use of carrageenans, more particularly kappa and iota carrageenans, obtained by the process of the invention in, but not limited to, pharmaceutical, food and industrial applications.

These carrageenans, more particularly kappa and iota carrageenans, are fully suitable, without further purification, for use in pharmaceutical, food and industrial products. However, if desired, it may also readily be subjected to further purification to produce a further purified carrageenan. Further purification may be performed by any known process suitable for this kind of product.

The present invention may be better understood with reference to the following examples.

### EXAMPLES

### Example 1

A 10 Kg. sample of dried Gigartina skottsbergii was washed in 100 liter of a water-based solution containing 2.2 Kg of Potassium Chloride at room temperature. After 30 minutes, seaweed was drained and manually sorted obtaining 9.3 grams of ‘sorted’ deaweed which contain Kappa II. These washed and sorted sample was manually chopped using scissors up to pieces of a size of 20-30 cm² and transferred to 100 l of a solution containing 0.3 grams of Sodium Dichloro-S-Triazintrione and 2.0 grams of Potassium Chloride. This reaction was held during 60 minutes at 12° C. De-colored product was then transferred to a container with 100 l of water at 55° C. containing 3 Kg of Potassium Chloride, where Sulfuric Acid was added up to pH 5.3. Product rest during 15 minutes. After this time 0.22 Kg of enzymes were added. The enzymes used had a ratio of 1:0.2 in terms of main activity (cellulase:xylanase).

This step took 4 hours at 50-55° C. under semi continuous agitation. During that time, pH was controlled at 5.0-5.5 adding Sulfuric Acid when necessary.

Product was drained during 15 minutes and then submerged in a water-based solution containing 5 Kg of Potassium Hydroxide and 5 Kg of Potassium Chloride, pre-heated at 75° C. Reaction was performed under semi-continuous agitation during 100 minutes, adding heat (indirect steam) to keep the temperature in 75° C.

Modified product was rinsed in fresh water at room temperature during 5 minutes. After this time the product was drained during 15 minutes and placed in a solution containing 2.5 Kg of KCl and where 18 mL of Sulfuric Acid was added and maintained at 40° C. during 35 minutes, time in which pH 7 was reached.

Neutralized product was transferred to a 40° C. solution containing 5 Kg of Potassium Chloride and 1.0 l of Sodium Hypochlorite. After 25 minutes, product was drained and washed with a solution containing 2 Kg of Potassium Chloride at 10° C. for 10 minutes and feed to a screw press at 10 rpm. Moisture of the product after this step was 68%. Later on, product was pelletized into stripes and dried in a conventional lab oven with air circulation.

Finally the product was grinded to a particle size of less than 150 μm.

A total of 5.7 g were recovered from the process, so yield over incoming seaweed was 56.8%.

Carrageenan obtained through this method has the following properties: Moisture content 4.5%, Ashes 33.3%, AIM 1.1%, pH 8.9, Viscosity 73 cP, Gel strength 180 g*cm⁻².

Determination of Moisture:

Here, moisture was determined gravimetrically, according to the following procedure: Using a spatula, 2 g of sample (P2) were weighted in a balance sensitive to +0.01 g in a porcelain crucible previously dried at 105° C. for 2 hours, kept in desiccator and weighted (P1). Sample was dried in stove at 105° C. during 2 hours. Then, crucible was removed from stove and kept in desiccator with silica-gel until room temperature (at least 20 minutes) and weight recorded.

Later on, crucible was returned to stove for one hour, removed and cooling down in desiccator. Weighed again. This operation was done until a constant weight (+0.002 g) was obtained (P3).

Moisture value was calculated according to formula:

% Moisture=1−((P3−P1)/P2)×100

Moisture value was expressed with two decimal numbers.

After moisture determination crucible with dehydrated sample was stored in desiccator.

Determination of the Ashes:

Total ashes were determined like residual weight after calcination, as follows:

Once moisture was already determined, crucible was brought to muffle furnace at 550° C. for 5 hours. After that time, waited until temperature dropped to 300° C. Then crucible was removed and kept it in desiccator until room temperature (at least 40 minutes). Sample was removed from desiccator and weighted (P4). Ashes value was calculated according to formula:

% Total Ashes=(P4−P1)/P2×100

Where, P1=Dry crucible initial weight, P2=Sample weight on dry basis, P4=Crucible final weight with calcined sample.

Ashes value was expressed with two decimal numbers.

Determination of AIM:

Acid Insoluble Matter (AIM), was determined gravimetrically as follows:

2 g sample was weighted into a 250 mL beaker using an analytical balance sensitive to +0.1 mg where 150 mL of deionized water were gently added. Then 15 mL of sulfuric acid 10% (by volume) was added. Beaker was covered with aluminum foil, sealing edges around the rim and all the mixture was heated up to 95° C. in a water bath thermostatically controlled. After 6 hours of digestion, sample was filtered using a previously dried glass fiber filter paper Toyo®GA55 for 1.6 μm and a glass funnel.

After filtration, residue of sample was washed with at least 150 mL of hot (90-95° C.) deionized water. Then, filter paper and its content were carefully placed in a porcelain crucible previously dried and weighted. Dried in oven at 105° C. during at least 3 hours, cooled 1 hour in a sealed desiccator, and weighted.

Acid insoluble matter was calculated as the difference between the weight of the filter paper and that of the residue.

AIM value was expressed with two decimal numbers.

Determination of Viscosity:

In an 800 mL beaker, 7.5 g of dry sample were weighed using a balance sensitive to ±0.01 g. Then, 500 mL of distilled water were measured in graduated cylinder and slowly added over sample while stirring with spatula. Later, beaker was introduced into a water bath thermostatically controlled at 90° C. After 20 minutes sample was agitated using a mixer. Once complete dissolution have been reached, beaker was removed from bath and temperature set at 75° C. Immediately viscosity was measured in a Brookfield® LV Rotational viscometer at 60 rpm speed with appropriated spindle according to the following table:

0-100 cP Spindle #1 (61)

101-500 cP Spindle #2 (62)

501-2000 cP Spindle #3 (63)

2001-10.000 cP Spindle #4 (64)

Viscosity was read after 15 seconds since the rotation was initiated. Result of viscosity was registered in whole numbers.

After measurement sample was stored and covered with watch glass for 24 hours at 20° C. for Gel Strength measurement.

Determination of Gel Strength:

Gel strength was determined as the force required breaking the gel as follows: After 24 hours storing at 20° C. Gels were separated from beaker sides with spatula, removed from beaker and inverted. Gel Strength was determined using a Stable Microsystems® TA XT2.1 Texture analyzer with 1.0 cm. diameter flat-base plunger. Plunger speed for this method was 1.6 mm*s⁻¹. Gel was placed on the base of the equipment, under the plunger and measurement started making three concentric measurements at halfway between the edge and center of the gel. Values were registered and the average calculated.

Result of gel strength was recorded in g*cm⁻² and whole numbers.

Determination of pH:

pH was measured using a pH-meter inserting the electrode Orion®9165 in the same Gel obtained from the solution prepared for viscosity and gel strength determination. Temperature was set to 25° C. and reading took after stabilization. pH value was expressed with one decimal number.

### Example 2

A 100 g sample of dried Eucheuma cottonii was treated according to the process described herein, as follows:

Sample was soaked/washed during 90 minutes in a tank containing 1.0 l of a solution having 13 g of Potassium Chloride at room temperature.

Later on, the product was directly placed in a beaker containing 25 g of Potassium Chloride and 1.5 g of Sodium Dichloro-S-Triazintrione at 25° C.

After 30 minutes the product was transferred to other beaker containing 25 g of Potassium Chloride at 60° C. where Sulfuric Acid was added up to reach pH 4.9, which occurred after 13 minutes.

Next, the product was immersed in a solution containing 4.0 g of a mix of Cellulase and Xylanase in a 1:0.1 ratio. This treatment was held 5 hours at 55° C., under semi continuous agitation.

Product was drained during 15 minutes and then submerged in a solution containing 90 g of Potassium Hydroxide and 50 g of Potassium Chloride, with alternating agitation at 77° C. After 120 minutes, the product was drained 15 minutes and put in a beaker containing fresh water during 10 minutes.

Rinsed product was then immersed in 1 l of a solution containing 20 g of Potassium Chloride, where was neutralized up to pH 7 with Sulfuric acid.

Later on, product was transferred to a solution containing 50 g of Potassium Chloride. Then 50 mL of Sodium Hypo-chlorite was added. Reaction lasts 25 minutes.

Bleached product was washed in a solution containing 20 g of Potassium Chloride during 20 minutes, and then pressed in a screw press. Moisture of the product at this step was 71%.

Later the product was extruded using a twin extruder with a screw diameter of 50 mm and a barrel length of 750 mm. The die was constituted of 3 holes of 6 mm. Conditions were 90° C. at 75 PSI and 500 rpm.

Extruded product was dried in a conventional lab oven with air circulation.

Finally the product was grinded up to less than 150 μm.

Yield in this example was 28.34% over incoming seaweed.

Carrageenan obtained through this method has the following properties: Moisture 7.08%, Ashes 30.7.3%, AIM 1.92%, pH 9.1, Viscosity 25 cP, Gel strength 542 g*cm−2. These test were performed in the same way like in Example 1.

### Example 3

50 Kg of wet (80% moisture) Sarcothalia crispata were treated in accordance with almost the same procedure described in Example 1, as follows:

Sample was washed in 100 liter of a water-based solution containing 5 Kg of Potassium Chloride at room temperature. After 30 minutes, seaweed was drain and manually sorted obtaining 43.0 grams of ‘sorted’ Kappa II containing seaweed. These washed and sorted sample was manually chopped using scissors up to pieces of a size of 40-50 cm2 and transferred to a solution containing 300 g of Sodium Dichloro-S-Triazintrione and 3.0 Kg of Potassium Chloride in 100 l of tap water. This reaction was held during 60 minutes at 12° C. De-colored product was then transferred to a container with 100 l of water at 50° C. containing 3 Kg of Potassium Chloride, where Sulfuric Acid was added up to pH 5.3 and left to rest during 15 minutes. After this time 220 g of enzymes were added. The enzymes used had a ratio of 1:0.2 in terms of main activity (cellulase:xylanase). This step took 5 hours at 50-55° C. under semi continuous agitation. During that time, pH was maintained at 5.2-5.5 dropping Sulfuric Acid when necessary.

After treatment product was drained during 15 minutes and submerged in a water-based solution containing 4 Kg of Potassium Hydroxide and 8 Kg of Potassium Chloride, pre-heated at 75° C. Reaction is performed under semi-continuous agitation during 75 minutes, adding indirect heat to keep the temperature in 75° C.

Modified product was rinsed in fresh water at room temperature during 5 minutes. After this time the product was drained during 15 minutes and placed in a solution containing 3.5 Kg of KCl and where 15 mL of Sulfuric Acid was added and maintained at 40° C. during 24 minutes, time where the pH 7 was reached.

Neutralized product was transferred to a 40° C. solution containing 5 Kg of Potassium Chloride and 1.2 l of Sodium Hypo-chlorite. After 30 minutes, product was drained and washed with a solution containing 3 Kg of Potassium Chloride at 10° C. Left 10 minutes and feed to a screw press. Moisture of the product at this step was 70%.

Later on, product was pelletized into stripes and dried in a conventional lab oven with air circulation.

Finally the product was grinded up to less than 200 μm.

A total of 4.359 g were recovered from the process, so yield over incoming seaweed was 43.59% based on dry seaweed.

Carrageenan obtained through this method has the following properties: Moisture 6.66%, Ashes 35.0%, AIM 1.32%, pH 9.4, Viscosity 81 cP, Gel strength 66 g*cm−2. These test were performed in the same way like in Example 1.

### Example 4

2 Kg of a mixture corresponding to the species Gigartina skottsbergii, Sarcothalia crispata and Eucheuma cottonii in a proportion of 40%, 20% and 40% respectively was submitted to the process detailed below.

The mixture was previously washed, sorted and chopped according to the initial steps of the processes described in Examples 1, 2 and 3, using 20 l when correspond.

Seaweed was submerged in a solution containing 50 g of Sodium Dichloro-S-Triazintrione and 700 g of Potassium Chloride in water at 15° C., during 50 minutes. After that time product was transferred to a solution containing 600 g of Potassium Chloride and where Sulfuric Acid have been previously added up to pH 4.7, where the product rested during of 15 minutes at 50° C.

Later on, the solution was heated to 55° C. and a preparation of enzymes containing a ratio of 1:0.15 Cellulase:Xylanase, granular form, in amount of 80 g was added and maintained under alternated agitation during 4.5 hours.

After enzymatic treatment, product was submerged in a solution containing 1,400 g of Potassium Hydroxide and 1,000 g of Potassium Chloride at 77° C. during 90 minutes; whit alternated agitation at 11 RPM. Then, product was drained during 15 minutes and then submerged in fresh water at room temperature during 7 minutes.

Rinsed product was submitted to neutralization in a reactor containing 800 g of Potassium Chloride in water at room temperature, where Sulfuric Acid was added up to pH 6.8.

Later on a final color removal was done, introducing the product into a bath containing 200 g of Sodium Hypo-chlorite and 600 g of Potassium Chloride during 25 minutes at 33° C.

Then product was transferred into a solution having 600 g of Potassium Chloride at 10-25° C. during 15 minutes.

Finally, product was pressed up to moisture 65%, pelletized to stripes of 0.9 cm in diameter, dried up to moisture content of 10% and grinded up to a size of less than 150 μm (75%) and 200 μm (100%).

Yield in this example was 48.02% over incoming seaweed, in dry basis.

Carrageenan obtained through this method has the following properties: Moisture 7.45%, Ashes 33.2%, AIM 1.74%, pH 9.2, Viscosity 44 cP, Gel strength 350 g*cm−2. These test were performed in the same way like in Example 1.

1. Process for producing carrageenans, more particularly kappa and iota carrageenans, containing less than 2% by weight of insoluble material, comprising the steps of:
i—preparing an aqueous suspension of a seaweed which contain carrageenans;
ii—reacting the resultant suspension with one or a mixture of enzyme(s).

2. Process according to claim 1, wherein the seaweed which contain carrageenans employed as the starting material may be chosen among Eucheuma cottonii, Eucheuma spinosum, Chondrus crispus, Gigartina stellata, Gigartina skottsbergii, Gigartina radula, Gymnogongrus furcellatus, Furcellaria fastigiata and Hypnea spp. Preferably, the seaweed is Eucheuma cottonii, Gigartina radula, Gigartina skottsbergii.

3. Process according to claims 1 or 2, wherein the aqueous suspension of step (i) is formed by mixing dried seaweed which contain carrageenans with a liquid, preferably aqueous.

4. Process according to any of claims 1 to 3, wherein the aqueous suspension of step (i) is obtained after:
washing and sorting of a carrageenan containing raw seaweed; and
optionally, chopping and/or bleaching the said seaweed.

5. Process according to any one of claims 1 to 4, wherein the content of seaweed dry matter in the suspension of step (i) is in the range of 5 to 20% by weight and preferably in the range of 10 to 15% by weight expressed by weight to weight.

6. Process according to any one of claims 1 to 5, wherein the enzymes have cellulase and/or xylanase activities.

7. Process according to any one of claims 1 to 6, wherein the enzymes, alone or in combination, having cellulase and/or xylanase activities are added in an amount up to about 15% by weight based on the weight of the seaweed which contain carrageenans.

8. Process according to claim 7, wherein the enzymes are added in an amount of about 5% by weight based on the weight of the seaweed which contain carrageenans.

9. Process according to any one of claims 1 to 8, wherein the reaction in step (ii) takes place at a pH of more or equal than 4 and no more than 6.

10. Process according to claim 9, wherein the reaction in step (ii) between the insoluble materials and the enzyme(s) proceeds at a pH of between 4.5 and 5.5.

11. Process according to any one of claims 1 to 10, wherein after the enzymatic treatment, the aqueous suspension of step (ii) is further subjected to alkali treatment.

12. Process according to claim 11, wherein the product resulting from the alkali treatment is then rinsed, neutralized, and optionally bleached.

13. Process according to claim 11, wherein the product is further subjected to the steps of washing, preferably dewatering, drying and finally size adjusting, the latter step being optional.

14. Process according to claim 13, wherein the carrageenan is dried to a dry matter content of at least 80% by weight, preferably at least 85% by weight, and more preferably at least 90% by weight.

15. Process according to claims 13 or 14, wherein after drying, further the carrageenan is processed by dry chopping and/or milling (ground) to a specific particle size.

16. Carrageenan, more particularly kappa and iota carrageenans containing less than 2% by weight of insoluble material obtained by a process according to any one of claims 1 to 16.

17. Use of carrageenans, more particularly kappa and iota carrageenans, obtained by the process according to any one of claims 1 to 16 in pharmaceutical, food and industrial applications.
